(19) United States
(12) Patent Application Publication
(10) Pub. No.: US 2020/0179643 A1
(43) Pub. Date: Jun. 11, 2020

(54) Catheter for Atraumatic Fluid Delivery

(71) Applicant: The Board of Regents of the University of Nebraska, Lincoln, NE (US)

(72) Inventors: Ioannis S. Chatzizisis, Omaha, NE (US); Benjamin S. Terry, Lincoln, NE (US); Sunandita Sarker, Lincoln, NE (US)

(21) Appl. No.: 16/631,732

(22) PCT Filed: Aug. 6, 2018
(86) PCT No.: PCT/US18/45432

§ 371 Date: Jan. 16, 2020

Related U.S. Application Data
(60) Provisional application No. 62/541,880, filed on Aug. 7, 2017.

(51) Int. Cl. A61M 25/00; A61M 25/09; A61M 5/178

(52) U.S. Cl. CPC A61M 25/003; A61M 25/09; A61M 5/178; A61M 2202/08; A61M 2205/0266; A61M 2025/0039; A61M 2025/0057

(57) ABSTRACT

A catheter for atraumatic delivery of fluid is disclosed. In embodiments, the catheter includes a catheter shaft with a guidewire lumen disposed within the catheter shaft and an infusion lumen at least partially defined by the catheter shaft. The infusion lumen may at least partially surround the guidewire lumen. The catheter shaft includes a plurality of pores extending through an outer surface of the catheter shaft to the infusion lumen. The plurality of pores are disposed near a distal end of the catheter shaft and are configured to radially dispense a fluid from the infusion lumen.

### CROSS-REFERENCE TO RELATED APPLICATIONS

The present application claims the benefit under 35 U.S.C. § 119(e) of U.S. Provisional Application Ser. No. 62/541,880, filed Aug. 7, 2017, and titled “Drug Delivery Catheter for Atherosclerosis Disease,” which is herein incorporated by reference in its entirety.

### BACKGROUND

Atherosclerosis is a chronic progressive cardiovascular disease associated with sub-endothelial accumulations of cholesterol and inflammatory cells. Identified as an inflammatory disease, atherosclerosis is open to several anti-inflammatory treatments, including targeted drug delivery with nano-carriers. Locally delivered lipid nanoparticles cross-link with specialized ligands against endothelial cell receptors to provide targeting capability to the plaque, potentially reducing inflammation and stabilizing the plaque.

Atherosclerotic Coronary Heart Disease (CHD) causes approximately 1 in 7 deaths. Of those 1 in 7 deaths, 75% are caused by ruptures of vulnerable plaques. Drug-eluting stents and drug-coated balloons are used to treat CHD; however, both devices have a high risk of rupturing the plaque built up in a biological lumen. Despite the risks, these treatments are often employed because stabilizing the plaque can reduce the possibility of a coronary event by up to 50%, or more in some cases. To reduce the risks associated with current methods of treating CHD, there is a need for methods of atraumatic delivery of therapeutic fluids to plaque built up in a biological lumen.

### SUMMARY

A catheter for atraumatic delivery of fluid is disclosed. In embodiments, the catheter includes a catheter shaft with a guidewire lumen disposed within the catheter shaft and an infusion lumen at least partially defined by the catheter shaft. The infusion lumen may at least partially surround the guidewire lumen. The catheter shaft includes a plurality of pores extending through an outer surface of the catheter shaft to the infusion lumen. The plurality of pores are disposed near a distal end of the catheter shaft and are configured to radially dispense a fluid from the infusion lumen.

A catheter system is also disclosed. In embodiments, the catheter system includes the catheter with a fluid delivery tube coupled to the infusion lumen and configured to direct the fluid from a fluid source into the infusion lumen. The catheter system can also include a guidewire that can be disposed within the guidewire lumen to deliver therapeutic agents or devices, and/or to position or manipulate the catheter.

A method for atraumatic delivery of fluid to a target within a biological lumen is also disclosed. In implementations, the method employs a catheter and/or catheter system as described herein. The method may include: introducing the catheter within a biological lumen; directing a fluid from a fluid source into the infusion lumen of the catheter; and radially dispensing the fluid from the infusion lumen in proximity to a target within the biological lumen through a plurality of pores formed near a distal end (e.g., near the tip) of the catheter.

This Summary is provided solely as an introduction to subject matter that is fully described in the Detailed Description and Drawings. The Summary should not be considered to describe essential features nor be used to determine the scope of the Claims. Moreover, it is to be understood that both the foregoing Summary and the following Detailed Description are example and explanatory only and are not necessarily restrictive of the subject matter claimed.

### BRIEF DESCRIPTION OF THE DRAWINGS

The detailed description is described with reference to the accompanying figures. The use of the same reference numbers in different instances in the description and the figures may indicate similar or identical items. Various embodiments or examples (“examples”) of the present disclosure are disclosed in the following detailed description and the accompanying drawings. The drawings are not necessarily to scale. In general, operations of disclosed processes may be performed in an arbitrary order, unless otherwise provided in the claims.

FIG. 1 is a schematic illustrating a catheter and a catheter system, in accordance with an example embodiment of the present disclosure.

FIG. 2A is a cross-sectional end view of a catheter, such as the catheter illustrated in FIG. 1, in accordance with an example embodiment of the present disclosure.

FIG. 2B is cross-sectional side view of a distal portion of a catheter, such as the catheter illustrated in FIG. 1, in accordance with an example embodiment of the present disclosure.

FIG. 3A is a side view showing a plurality of pores formed in a non-uniform (e.g., helical) arrangement at a distal portion of a catheter, such as the catheter illustrated in FIG. 1, in accordance with an example embodiment of the present disclosure.

FIG. 3B is a side view showing a plurality of pores formed in a non-uniform (e.g., multi-helical) arrangement at a distal portion of a catheter, such as the catheter illustrated in FIG. 1, in accordance with an example embodiment of the present disclosure.

FIG. 3C is a side view showing a plurality of pores formed in an offset arrangement at a distal portion of a catheter, such as the catheter illustrated in FIG. 1, in accordance with an example embodiment of the present disclosure.

FIG. 4A is a side view showing an example environment in which a catheter, such as catheter illustrated in FIG. 1, can be employed to dispense fluid in proximity to a target within a biological lumen, in accordance with an example embodiment of the present disclosure.

FIG. 4B is a side view showing an example environment in which a catheter, such as catheter illustrated in FIG. 1, can be employed to dispense fluid in proximity to a target within a biological lumen, in accordance with an example embodiment of the present disclosure.

FIG. 5A is a cross-sectional end view of a catheter with a guidewire lumen and an infusion lumen, in accordance with an example embodiment of the present disclosure.

FIG. 5B is a cross-sectional end view of a catheter with a guidewire lumen and an infusion lumen, in accordance with an example embodiment of the present disclosure.

FIG. 5C is a cross-sectional end view of a catheter with a combined guidewire and infusion lumen that can be selectively occluded by a deployable blocking element, in accordance with an example embodiment of the present disclosure.

FIG. 5D is a cross-sectional end view of a catheter with a combined guidewire and infusion lumen that can be selectively occluded by a deployable blocking element, in accordance with an example embodiment of the present disclosure.

FIG. 6 is a flow-diagram illustrating an example implementation of a method for atraumatic delivery of fluid to a target within a biological lumen with a catheter, such as the catheter illustrated in any of FIGS. 1 through 5D, or a combination thereof.

### DETAILED DESCRIPTION

Atherosclerosis is a cardiovascular disease in which plaque builds up inside arteries, and the plaque hardens such that the arteries are narrowed, limiting blood flow to organs and other parts of the body. Lipid nanoparticles, which contain anti-inflammatory molecules, can be used to reduce and/or stabilize the plaque. However, some delivery techniques (e.g., drug-eluting stents or drug-coated balloons) have a high risk of rupturing the plaque, which can be fatal in some cases. To avoid rupturing the plaque, it may be advantageous to deliver lipid nanoparticles and/or other therapeutic fluids to the plaque without making direct contact with the plaque itself.

A catheter for atraumatic delivery of fluid (e.g., lipid nanoparticles and/or other therapeutic fluids) is disclosed. The catheter can be used to treat atherosclerosis or for any other treatment or therapy that requires delivery of fluid to a target within a biological lumen (e.g., a blood vessel, intestine, ureter, airway, or the like). In embodiments of this disclosure, which are described in further detail below, the catheter includes a catheter shaft with a plurality of pores formed near a distal end of the catheter shaft. The catheter is configured to radially dispense a fluid through the pores in proximity to a target (e.g., plaque) within a biological lumen (e.g., blood vessel) without making direct contact with the target. For example, in some implementations, a tip of the catheter is guided past the target, and the pores are brought in proximity to (e.g., alongside or near (e.g., just ahead of or past)) the target so that the fluid dispensed through the pores can be directed to the target in a direction that is normal or substantially normal (e.g., at an angle in the range of 60 to 120 degrees) to the direction of biological fluid (e.g., blood) flow in the biological lumen.

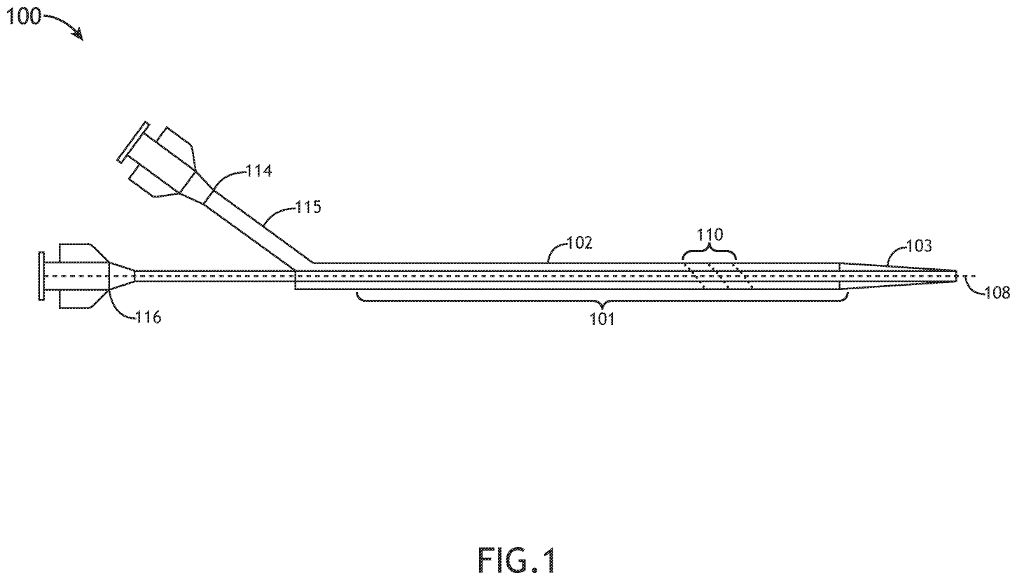

FIG. 1 illustrates a catheter system 100 in accordance with embodiments of the present disclosure. The catheter system 100 includes a catheter 101 configured to be at least partially disposed within a biological lumen. In embodiments, the catheter 101 may be formed from a biologically compatible material, such as, but not limited to, PEBAX, TEFLON, silicon, or any other plastic, elastomer, or combination thereof. The catheter 101 includes a catheter shaft 102 that defines a longitudinal body of the catheter 101. The catheter shaft 102 may be flexible and appropriately sized for insertion into a biological lumen. For example, the catheter shaft 102 may have a diameter in the range of 0.5 mm to 10 mm. In some embodiments, the catheter shaft 102 has a diameter of approximately 1 mm.

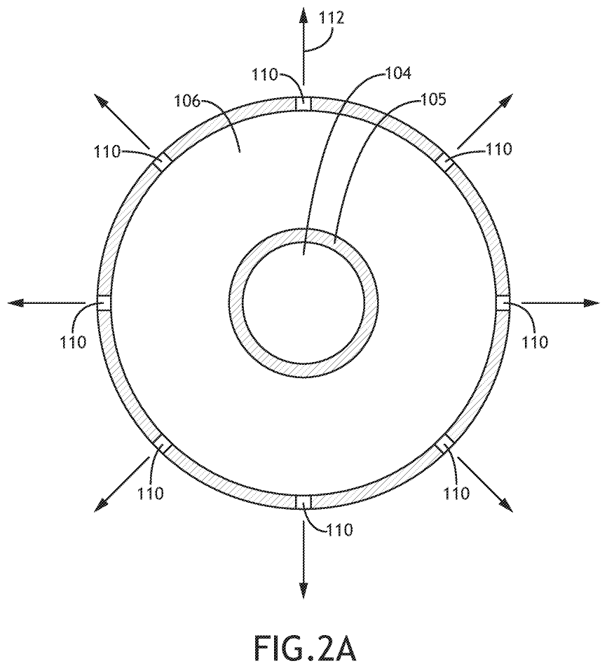

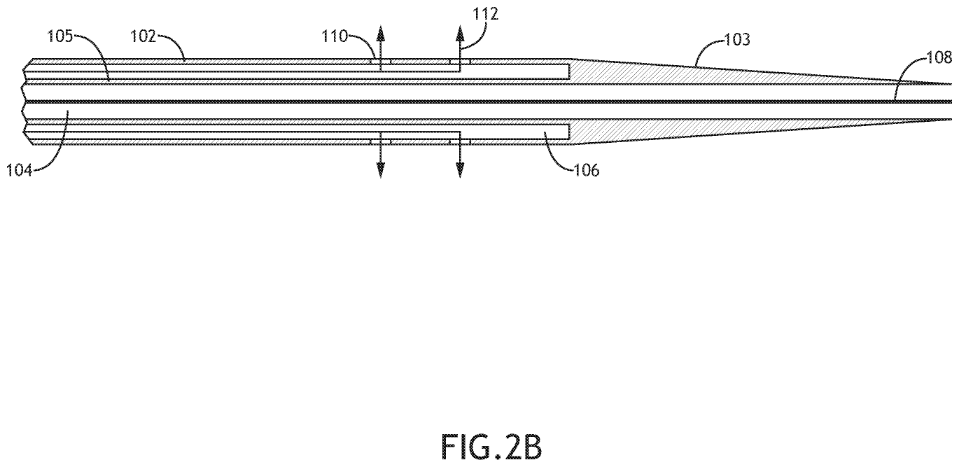

As shown in FIG. 2A, in embodiments, the catheter shaft 102 includes a guidewire lumen 104 disposed within the catheter shaft 102. In embodiments, the guidewire lumen 104 is at least partially defined by an inner wall 105 (or tube) that extends along or parallel to a longitudinal axis of the catheter shaft 102. The catheter shaft 102 and the guidewire lumen 104 can both be formed from a biologically compatible material, such as, but not limited to, PEBAX, TEFLON, silicon, or any other plastic, elastomer, or combination thereof. In some embodiments, the guidewire lumen 104 and the catheter shaft 102 are formed from the same material. In other embodiments, the guidewire lumen 104 is formed from a different material than the catheter shaft 102. The guidewire lumen 104 may have a cross-sectional area that is 50% or less than the cross-sectional area of the catheter shaft 102. For example, the guidewire lumen 104 may have a diameter in the range of 0.1 mm to 5 mm. In some embodiments, the guidewire lumen 104 has a diameter of approximately 0.4 mm.

The guidewire lumen 104 is configured to receive a guidewire 108 that can extend longitudinally through the guidewire lumen 104 to (and possibly out from) a tip 103 at the distal end of the catheter shaft 102. For example, the guidewire 108 may be fed into the guidewire lumen 104 through a guidewire entrance 116 (e.g., a tube or conduit) coupled to the guidewire lumen 104. The guidewire 108 may be formed from a biologically compatible material, such as, but not limited to, gold, nitinol, platinum, stainless steel, tungsten, or a combination thereof. In some embodiments, the guidewire 108 may be coated with a polymer, such as, but not limited to, silicone, tetrafluoroethylene (TFE), polytetrafluoroethylene (PTFE), or the like. The guidewire 108 may be appropriately sized for insertion into the guidewire lumen 104. For example, in embodiments the guidewire 108 may have a diameter in the range of 0.1 mm to 1 mm. In some embodiments, the guidewire 108 has a diameter of approximately 0.36 mm.

The catheter shaft 102 further includes an infusion lumen 106 configured to receive fluid (e.g., a therapeutic fluid and/or carrier fluid) for delivery to a target within a biological lumen. In embodiments, the catheter shaft 102 itself defines at least a portion of the infusion lumen 106. For example, the catheter shaft 102 may define at least a portion of an outer wall of the infusion lumen 106. The infusion lumen 106 may at least partially surround the guidewire lumen 104. For example, as shown in FIG. 2A, the guidewire lumen 104 may be disposed within the infusion lumen 106. Further, in some embodiments, the guidewire lumen 104 and the infusion lumen 106 may be concentric or coaxial. In some embodiments, the pressure inside the infusion lumen 106 is high enough for fluid 112 to be dispense in a controlled manner. For example, the pressure inside the infusion lumen 106 may in the range of 50 to 60 PSI.

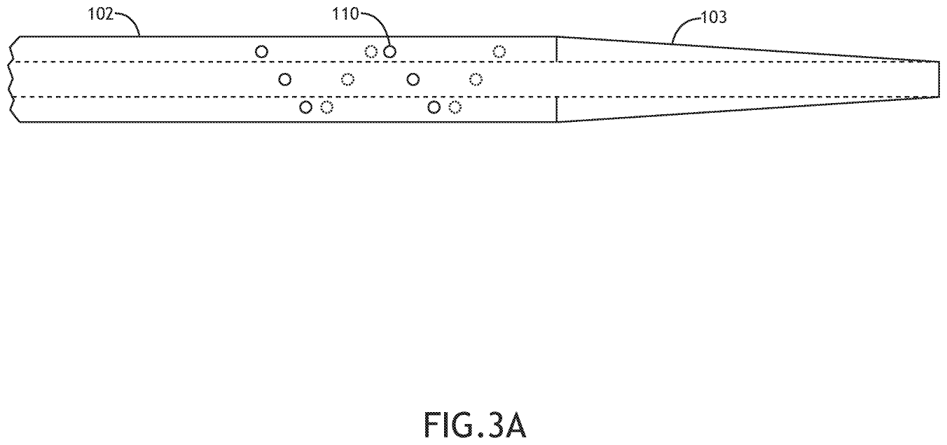

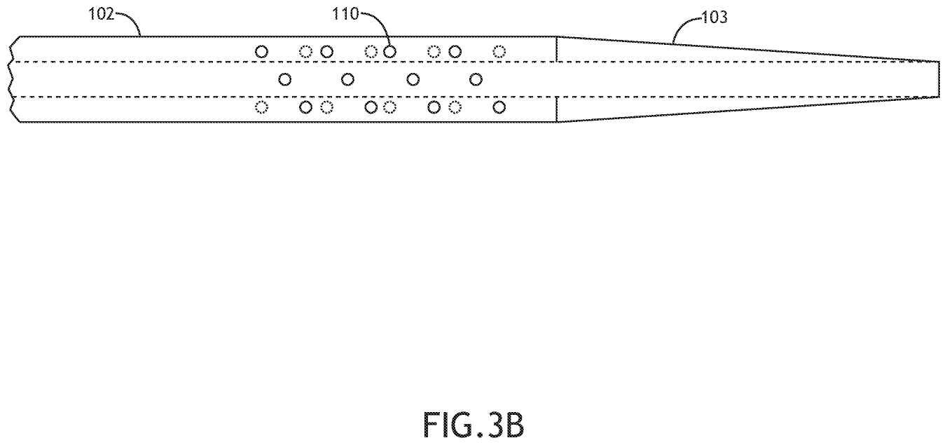

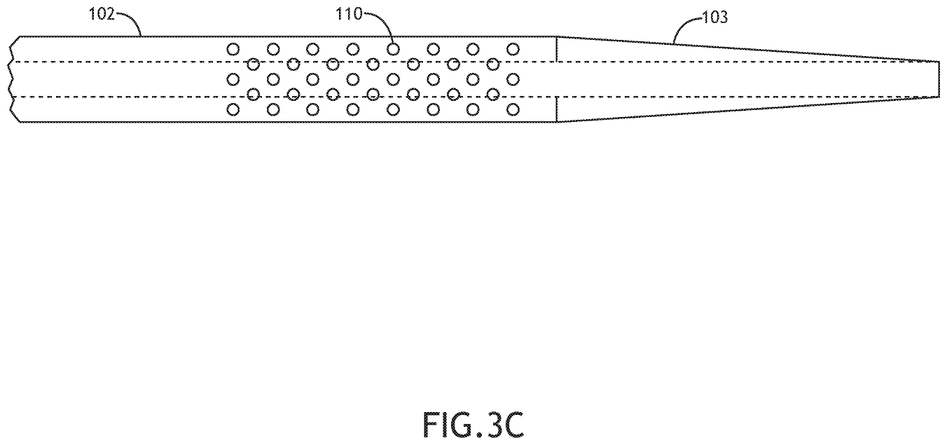

The catheter 101 is configured to dispense fluid from the infusion lumen 106 through a plurality of pores 110 disposed near a distal end of the catheter shaft 102 (e.g., along the body of the catheter shaft 102, near the tip 103). The pores 110 may be arranged about the longitudinal axis of the catheter shaft 102. For example, various arrangements are shown in FIGS. 2A through 3C and are described in further detail below. As shown in FIG. 2A, the pores 110 extend through an outer surface of the catheter shaft 102 to the infusion lumen 106 and are configured to radially dispense fluid 112 from the infusion lumen 106. Examples of fluid 112 that can be dispensed from the plurality of pores 110 include, but are not limited to, fluids including therapeutic agents, solutions including medications, contrast agents, or the like. In some embodiments, the fluid 112 includes lipid nanoparticles that may encapsulate a pre-selected drug (e.g., an anti-inflammatory drug). As shown in FIG. 1, the catheter system 100 may include a fluid delivery tube 115 coupled to the infusion lumen 106 and configured to direct the fluid 112 from a fluid source 114 into the infusion lumen 106. In embodiments, the fluid source 114 can include, but is not limited to, a syringe, an electromechanically actuated syringe, a fluid pump (e.g., peristaltic or pneumatic pump), any combination thereof, or the like.

In the embodiment shown in FIG. 2A, a cross-section of the catheter 101 shows six pores 110 uniformly arranged about the longitudinal axis of the catheter shaft 102 with a 60 degree angle of separation between adjacent pores 110. This configuration is provided for illustrative purposes and other distributes and/or numbers of pores can be employed without departing from the scope of this disclosure. For example, in another embodiment, the catheter shaft 102 may include four pores 110 that are uniformly arranged with a 90 degree angle of separation between adjacent pores 110. In other embodiments, the pores 110 may have different spatial distributes about the longitudinal axis of the catheter shaft 102 and/or longitudinally along the body of the catheter shaft 102. The pores 110 may have any angle of separation between adjacent pores 110 (e.g., including, but not limited to, an angle in the range of 30 to 90 degrees).

As shown in FIGS. 2A and 2B, the pores 110 are configured to dispense the fluid 112 radially from the catheter shaft 102. For example, the fluid 112 can be dispensed through the pores 110 in a direction that is normal or substantially normal (e.g., at an angle in the range of 60 to 120 degrees) to the direction of biological fluid (e.g., blood) flow in the biological lumen. In embodiments, the distal end of the infusion lumen 106 may be closed so that the fluid 112 cannot be dispensed from the tip 103 of the catheter 101 and therefore must exit the pores 110. The pores 110 may be located at a distance from the tip 103. For example, the pores 110 may be in the range of 0.5 to 3 cm from the tip 103. In some embodiments, the pores 110 are located about 1 cm from the tip.

In some embodiments, the pores 110 have respective diameters in the range of 15 to 25 micrometers. For example, in an embodiment, each pore 110 has a diameter of approximately 20 micrometers. Embodiments of the disclosure may adapt various pore diameters to accommodate various environments and applications for the catheter 101. In some embodiments, the flow velocity is adjusted based on the average pore diameter of catheter 101 so that the volume flow rate is in the range of 1 to 5 ml/min. For example, in an embodiment, the catheter system 100 is configured to dispense fluid 112 at a flow rate of approximately 2 ml/min.

In some embodiments, the pores 110 are arranged about the longitudinal axis of the catheter shaft 102 such that a group of pores 110 is in one plane (e.g., forming a circle or ellipse about the longitudinal axis of the catheter shaft 102). In other embodiments, the pores 110 may be arranged non-uniformly or according to different geometry to control the fluid flow from the pores 110 and/or to maintain structural integrity of the catheter shaft 102. For example, FIGS. 3A through 3C show side views of a distal portion of the catheter 101 with different pore arrangements, in accordance with various embodiments of the present disclosure.

The embodiments in FIGS. 3A through 3C employ non-uniform pore arrangements that have a reduced number of pores 110 per cross-sectional plane of the catheter shaft 102. Such arrangements can be employed to prevent the pores 110 from acting as a perforation about the catheter shaft 102 that may be prone to ripping or tearing. In an embodiment shown in FIG. 3A, the pores 110 are arranged non-uniformly (e.g., helically) about the longitudinal axis of the catheter shaft 102. In some embodiments, the pores 110 may define multiple helixes (e.g., 2, 3, or more helixes). For example, FIG. 3B shows another embodiment of the catheter 101, where the pores 110 are disposed in a non-uniform (e.g., double-helix) arrangement about the longitudinal axis of the catheter shaft 102. In other embodiments, the pores 110 can be offset from one another. For example, in an offset pattern or arrangement, such as the arrangement shown in FIG. 3C, a first group of pores 110 can be arranged along the circumference of the catheter shaft 102 (in one cross-sectional plane), and an adjacently disposed second group of pores 110 may be arranged along the circumference of the catheter shaft 102 (in another cross-sectional plane) at an offset (e.g., a 10 to 90 degree offset) such that the first group of pores 110 does not align with the second group of pores 110. As shown in FIG. 3C, this pattern can be repeated a number of times along a distal portion of the catheter shaft 102.

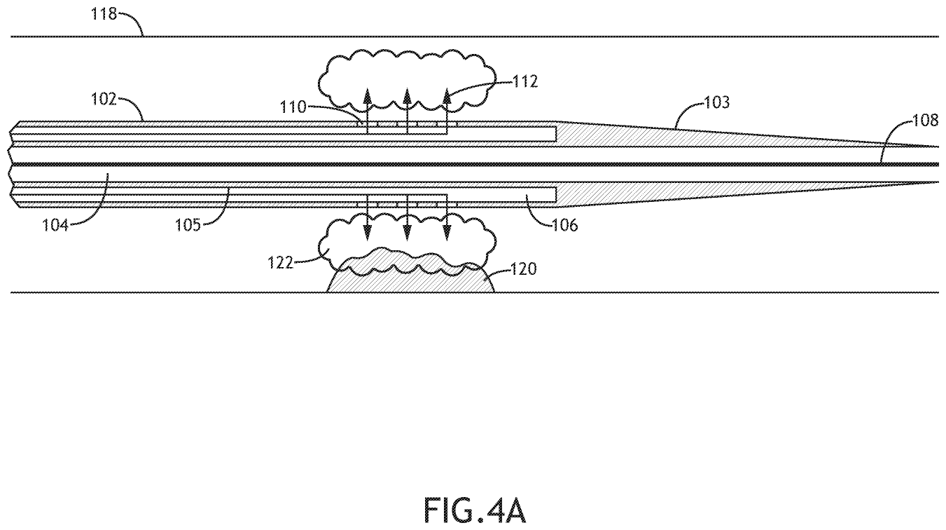

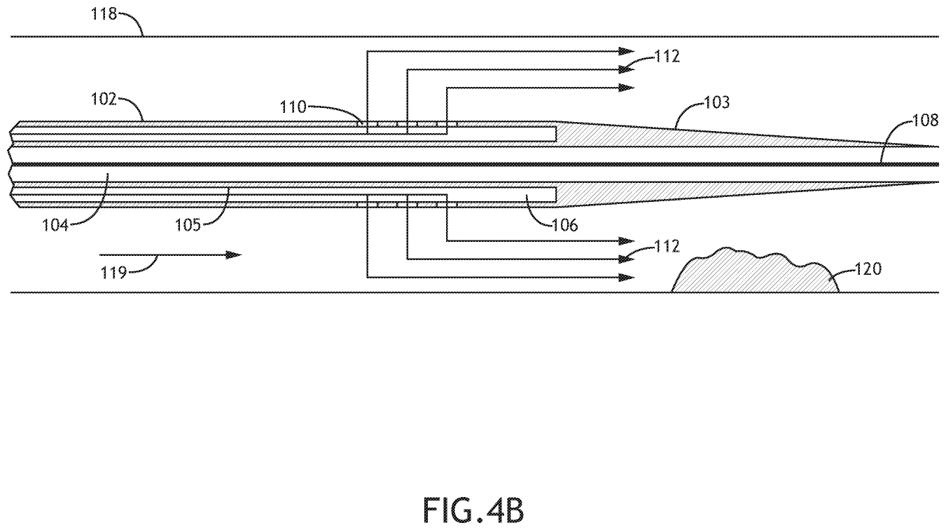

FIGS. 4A and 4B illustrate an example environment in which the catheter 101 may be deployed. The catheter 101 may be inserted within a biological lumen 118 (e.g., a blood vessel, intestine, ureter, airway, or the like). For example, the catheter 101 may be configured for insertion within an artery. In some embodiments, the catheter 101 is configured for insertion with a stenotic artery. The guidewire 108 disposed within the catheter shaft 102 may assist in guiding the catheter 101 to a target 120 (e.g., plaque) in the biological lumen 118. In use, the catheter tip 103 may be directed to a site in proximity to the target 120 so that the pores 110 on the catheter shaft 102 are brought in proximity to (e.g., adjacent to or near (e.g., just ahead of or past)) the target 120. The fluid source 114 can then direct fluid 112 into the infusion lumen 106 so that the pores 110 radially dispense the fluid 112 from the infusion lumen 106 in proximity to (e.g., directed at or near) the target 120 in the biological lumen 118. Meanwhile, the tip 103 of the catheter 101 does not make physical contact with the target 120. In some implementations, contact with the target 120 is completely or substantially avoided. In this regard, the catheter 101 is configured for atraumatic delivery of the fluid 112 to the target 120. The fluid 112 is radially dispensed from the pores 110 in a controlled manner. For example, in some embodiments, the pore distribution and flow rate cause the fluid 112 to form a radial stream or cloud 122 in proximity to the target 120 so that one or more active agents (e.g., therapeutic agents, diagnostic agents, etc.) in the fluid 112 can be dispersed upon and/or absorbed by the target 120. As shown in FIG. 4B, in some embodiments, the pores 110 achieve laminar flow of the fluid 112 in radially dispensed streams that are carried by the biological fluid 119 to the target 120. In such an implementation, the catheter 101 may be guided to a position within the biological lumen 118 where the pores 110 are a small distance (e.g., 0.1 to 10 cm) from the target 120.

In an example use case, the catheter 101 is guided to diseased plaque in the stenotic artery, and the fluid source 114 then directs lipid nanoparticles into the infusion lumen 106. The pores 110 can then radially dispense the lipid nanoparticles in proximity to the plaque. The fluid 112 may form a radial stream or cloud 122 of lipid nanoparticles in proximity to the plaque so that the lipid nanoparticles can be dispersed upon and/or absorbed by the plaque to stabilize plaque. Meanwhile, the catheter tip 103 does not make physical contact with the diseased plaque. In some implementations, no portion of the catheter 101 makes direct contact with the plaque, thereby preventing a possible rupturing of the plaque.

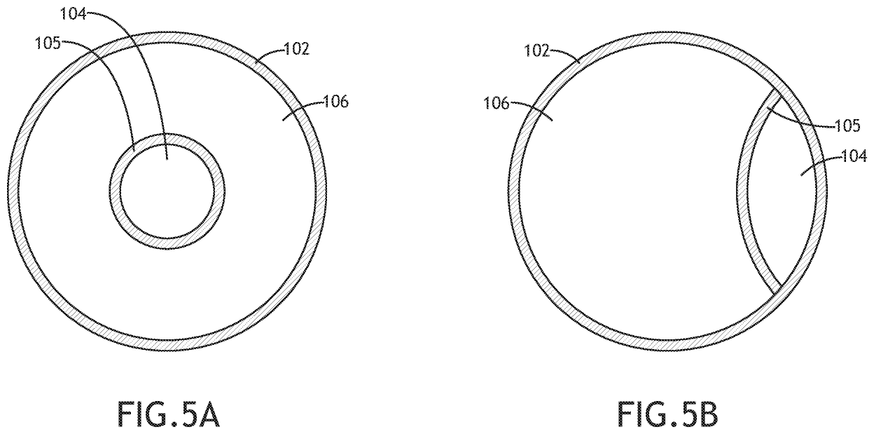

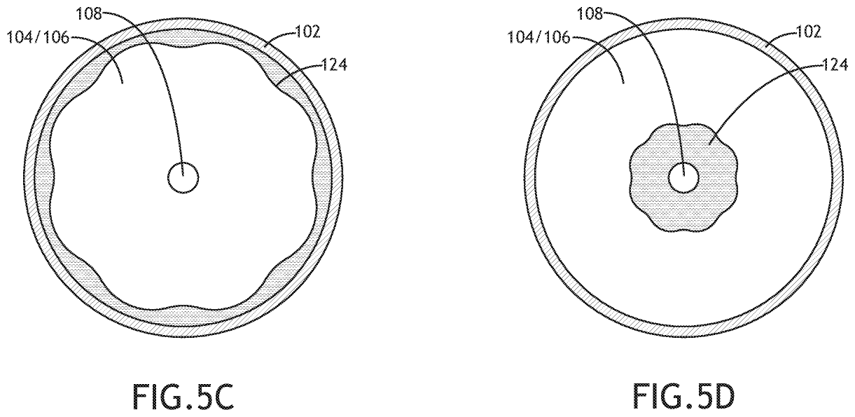

FIGS. 5A through 5D show cross-sectional end views of the catheter 101 in accordance with various embodiments of the disclosure, In embodiments, the guidewire lumen 104 and the infusion lumen 106 are coaxial or parallel to one another. For example, as shown in FIG. 5A, the guidewire lumen 104 and infusion lumen 106 may create a concentric dual lumen. In such embodiments, the infusion lumen 106 completely surrounds the guidewire lumen 104 such that the inner wall 105 of the infusion lumen 106 defines the outer wall 105 of the guidewire lumen 104. In other embodiments (e.g., as shown in FIG. 5B), the guidewire lumen 104 and the infusion lumen 106 create an eccentric dual lumen. In such embodiments, the infusion lumen 106 is adjacent to and may partially surround the guidewire lumen 104. The catheter shaft 102 may define a portion of the infusion lumen 106 and a portion of the guide wire lumen 104 with a shared inner wall 105 separating the two lumens.

In any of these embodiments, the infusion lumen 106 may have a closed distal end, and the guidewire lumen 104 may have an open distal end. The distal end of the infusion lumen 106 can be blocked or closed off so that the fluid 112 is primarily (or only) released from the pores 110. Meanwhile, the distal end of the guidewire lumen 104 can allow the guide wire 108 to travel through an opening at the tip 103 of the catheter 101.

In some embodiments, the catheter system 100 includes a blocking element that can selectively occlude the infusion lumen 106 and/or tip 103 of the catheter 101. For example, as shown in FIG. 5C, a deployable blocking element 124 (e.g., a balloon) may be coupled to an inner surface of the catheter shaft 102 (or infusion lumen 106). In another example embodiment that is shown in FIG. 5D, the deployable blocking element 124 is coupled to a distal end of the guidewire 108. The deployable blocking element 124 can be selectively deployed (e.g., deflated or mechanically actuated) so that the deployable blocking element 124 occludes the lumen in which it is disposed.

As shown in FIGS. 5C and 5D, separate guidewire and infusion lumens 104 and 106 are not required when the catheter system 100 includes a deployable blocking element 124 configured to selectively occlude the distal end (e.g., tip 103) of the catheter 101. Instead, the shaft 102 can define a combined guidewire and infusion lumen 104/106 where the guidewire 108 and the fluid 112 can both be directed through the combined lumen 104/106, and the fluid 112 can be forced out through the pores 110 formed in the catheter shaft 102 by first deploying the deployable blocking element 124 to occlude the distal end/tip 103 of the catheter 101 and then directing fluid 112 through the combined lumen 104/106.

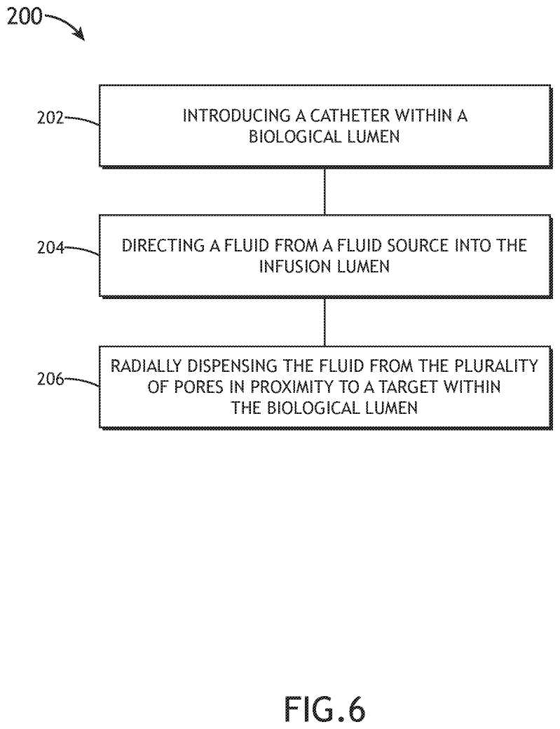

FIG. 6 illustrates an example implementation of a method 200 that employs the catheter system 100 for atraumatic delivery of fluid to a target within a biological lumen. In general, operations of disclosed processes (e.g., method 200) may be performed in an arbitrary order, unless otherwise provided herein.

The method 200 includes introducing the catheter 101 within a biological lumen 118 (block 202). For example, as shown in FIGS. 4A and 4B, the catheter 101 can be inserted into the biological lumen 118. In implementations, the tip 103 of the catheter 101 may be directed past a target 120 (e.g., plaque) in the biological lumen 118 without making physical contact between the tip 103 and the target 120.

Fluid is then directed from a fluid source 114 into the infusion lumen 106 (block 204). For example, as shown in FIG. 1, the fluid delivery tube 115 or a portion of the infusion lumen 106 itself may be used to direct fluid 112 from the fluid source 114 into the infusion lumen 106. In implementations, the fluid source 114 can include, but is not limited to, a syringe, an electromechanically actuated syringe, a fluid pump (e.g., peristaltic or pneumatic pump), any combination thereof, or the like.

The fluid 112 is radially dispensed from pores 110 formed near a distal end of the catheter 101 (block 206). For example, the fluid 112 can be dispensed from the pores 110 in proximity to (e.g., adjacent to or near (e.g., just ahead of or past)) the target 120 within the biological lumen 118. As shown in FIGS. 4A and 4B, the fluid 112 may be radially dispensed from the pores 110 in a controlled manner. For example, in some embodiments, the pore distribution and flow rate cause the fluid 112 to form a radial stream or cloud 122 in proximity to the target 120 so that one or more active agents (e.g., therapeutic agents, diagnostic agents, etc.) in the fluid 112 can be dispersed upon and/or absorbed by the target 120. In implementations, the pores 110 are configured to dispense the fluid 112 in a direction that is normal or substantially normal (e.g., at an angle in the range of 60 to 120 degrees) to the direction of biological fluid (e.g., blood) flow in the biological lumen 118. Flowing the fluid 112 in a direction that is normal or substantially normal to the direction of biological fluid flow can help avoid having too much fluid pressure/force on diseased plaque or any other target 120 in the biological lumen 118 (e.g., from a combination of biological fluid and dispensed fluid 112 flowing in the same direction), which may result in unwanted rupture or dislodging of the plaque.

The method 200 may further include any step or operation implied or required by the embodiments of catheter system 100 described herein. The catheter system 100 can also include any additional component or functionality expressed or implied by the method 200.

Although the technology has been described with reference to the embodiments illustrated in the attached drawing figures, equivalents may be employed and substitutions made herein without departing from the scope of the technology as recited in the claims. Components illustrated and described herein are merely examples of a device and components that may be used to implement the embodiments of the present invention and may be replaced with other devices and components without departing from the scope of the invention. Furthermore any dimensions, degrees, and/or numerical ranges provided herein are to be understood as non-limiting examples unless otherwise specified in the claims.

What is claimed is:

1. A catheter, comprising:
a catheter shaft;
a guidewire lumen disposed within the catheter shaft;
an infusion lumen at least partially defined by the catheter shaft, the infusion lumen at least partially surrounding the guidewire lumen; and
a plurality of pores extending through an outer surface of the catheter shaft to the infusion lumen, the plurality of pores disposed near a distal end of the catheter shaft and configured to radially dispense a fluid from the infusion lumen.

2. The catheter of claim 1, further comprising:
a fluid delivery tube coupled to the infusion lumen and configured to direct the fluid from a fluid source into the infusion lumen.

3. The catheter of claim 2, wherein the fluid source is a syringe.

4. The catheter of claim 1, wherein the plurality of pores are arranged about an axis of the catheter shaft.

5. The catheter of claim 4, wherein the plurality of pores are arranged non-uniformly about the axis of the catheter shaft.

6. The catheter of claim 4, wherein the plurality of pores are uniformly arranged about the axis of the catheter shaft with an angle of separation in the range of 30 degrees to 90 degrees between adjacent pores.

7. The catheter of claim 1, wherein the infusion lumen and the guidewire lumen are coaxial.

8. A system for atraumatic delivery of fluid to a target within a biological lumen, comprising:
a guidewire;
a catheter shaft;
a guidewire lumen disposed within the catheter shaft and configured to receive the guidewire;
an infusion lumen at least partially defined by the catheter shaft, the infusion lumen at least partially surrounding the guidewire lumen;
a fluid delivery tube coupled to the infusion lumen and configured to direct a fluid from a fluid source into the infusion lumen; and
a plurality of pores extending through an outer surface of the catheter shaft to the infusion lumen, the plurality of pores disposed near a distal end of the catheter shaft and configured to radially dispense the fluid from the infusion lumen in proximity to a target within a biological lumen.

9. The system of claim 8, wherein the fluid comprises a therapeutic agent.

10. The system of claim 9, wherein the therapeutic agent comprises a lipid nanoparticle.

11. The system of claim 8, wherein the fluid source is a syringe.

12. The system of claim 8, wherein the plurality of pores are arranged about an axis of the catheter shaft.

13. The system of claim 12, wherein the plurality of pores are arranged non-uniformly about the axis of the catheter shaft.

14. The system of claim 12, wherein the plurality of pores are uniformly arranged about the axis of the catheter shaft with an angle of separation in the range of 30 degrees to 90 degrees between adjacent pores.

15. The system of claim 8, wherein the infusion lumen and the guidewire lumen are coaxial.

16. A method for atraumatic delivery of fluid to a target within a biological lumen, comprising:
introducing a catheter within a biological lumen, the catheter including: a catheter shaft; a guidewire lumen disposed within the catheter shaft; an infusion lumen at least partially defined by the catheter shaft, the infusion lumen at least partially surrounding the guidewire lumen; and a plurality of pores extending through an outer surface of the catheter shaft to the infusion lumen;
directing a fluid from a fluid source into the infusion lumen; and
radially dispensing the fluid from the plurality of pores in proximity to a target within the biological lumen.

17. The method of claim 16, wherein the plurality of pores are arranged about an axis of the catheter shaft.

18. The method of claim 17, wherein the plurality of pores are arranged non-uniformly about the axis of the catheter shaft.

19. The method of claim 17, wherein the plurality of pores are uniformly arranged about the axis of the catheter shaft with an angle of separation in the range of 30 degrees to 90 degrees between adjacent pores.

20. The method of claim 16, wherein the infusion lumen and the guidewire lumen are coaxial.
